(19) United States
(12) Patent Application Publication
(10) Pub. No.: US 2025/0001103 A1
(43) Pub. Date: Jan. 2, 2025

(54) PORTABLE NEBULIZER AND SHUTDOWN METHOD OF PORTABLE NEBULIZER

(71) Applicant: HCMed Innovations Co., LTD., Taipei City (TW)

(72) Inventors: YEN-TING CHEN, Taipei City (TW); CHIEN-SHEN TSAI, New Taipei City (TW); CHUN-CHIA JUAN, New Taipei City (TW)

(21) Appl. No.: 18/757,711

(22) Filed: Jun. 28, 2024

Related U.S. Application Data
(60) Provisional application No. 63/524,197, filed on Jun. 29, 2023.

(30) Foreign Application Priority Data
Jun. 14, 2024 (CN) 202410764090.5

(51) Int. Cl. A61M 11/00

(52) U.S. Cl. CPC A61M 11/005; A61M 2205/3327; A61M 2205/3389; A61M 2205/50

(57) ABSTRACT

A portable nebulizer and a shutdown method of the portable nebulizer are provided. The portable nebulizer includes a medicine storage device, a sensing device, a nebulization device, a driving device and a processing device. The medicine storage device is configured to contain predetermined medical fluid. The sensing device is located in the medicine storage device. The nebulization device is configured to nebulize the predetermined medical fluid and simultaneously transmit a sensing signal to the sensing device. The driving device is electrically connected to the nebulization device and transmits a driving signal to the nebulization device. The processing device is electrically connected to the sensing device and the driving device, and collects electrical information of the sensing signal and electrical information of the driving signal to calculate medical fluid volume indication information.

### CROSS-REFERENCE TO RELATED PATENT APPLICATION

This application claims the benefit of priority to China Patent Application No. 202410764090.5, filed on Jun. 14, 2024, in the People's Republic of China. The entire content of the above identified application is incorporated herein by reference.

This application claims the benefit of priority to the U.S. Provisional Patent Application Ser. No. 63/524,197, filed on Jun. 29, 2023, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a nebulizer and a control method of the nebulizer, and more particularly to a portable nebulizer and a shutdown method of the portable nebulizer.

### BACKGROUND OF THE DISCLOSURE

In recent years, with the increase of the severity of air pollution, more and more people have respiratory diseases. For treating respiratory diseases, spray treatment has become a relatively popular method in recent years.

The nebulization methods currently used in medical-grade portable nebulizers can be generally divided into pneumatic or ultrasonic types, which can convert the medical liquid into aerosol particles having sizes of 3 microns to 5 microns, such that inhaling aerosol fluid is used to treat the bronchial tubes and lungs. Since the aerosol fluid is inhaled through the mouth and nose of a user, enters the bronchus, and then spreads to the alveoli in the lungs, the spray treatment method can allow the aerosol fluid to reach the location of the lesion for direct treatment. Compared with a traditional manner of taking oral medications, the inhaled drug takes effect more quickly.

However, there is still room for improvement in the use of conventional portable nebulizers. For example, when the predetermined medical liquid in the medicine placement device of the portable nebulizer has been used up, and the predetermined medical liquid in the medicine placement device produces a lot of foam due to its medicinal properties during the process of being converted into aerosol, the processing device of the portable nebulizer may misjudge that there is still the predetermined medical liquid in the medicine placement device, thus causing the nebulizing device of the portable nebulizer to continuously vibrate. If the nebulizer device often continues to vibrate when the predetermined medicinal liquid has been used up, the service life of the nebulizer device will be reduced or the nebulizer device will be directly damaged.

### SUMMARY OF THE DISCLOSURE

In response to the above-referenced technical inadequacies, the present disclosure provides a portable nebulizer and a shutdown method of the portable nebulizer.

In order to solve the above-mentioned problems, one of the technical aspects adopted by the present disclosure is to provide a portable nebulizer. The portable nebulizer includes a medicine storage device, a sensing device, a nebulization device, a driving device, and a processing device. The medicine storage device is configured to contain predetermined medical fluid. The sensing device is located in the medicine storage device. The nebulization device is configured to nebulize the predetermined medical fluid and simultaneously transmit a sensing signal to the sensing device. The driving device is electrically connected to the nebulization device and transmits a driving signal to the nebulization device. The processing device is electrically connected to the sensing device and the driving device, and collects electrical information of the sensing signal and electrical information of the driving signal to calculate and generate medical fluid volume indication information. The processing device is configured to determine whether or not to execute a shutdown procedure according to a relationship between the medical fluid volume indication information and a predetermined value range.

In order to solve the above-mentioned problems, another one of the technical aspects adopted by the present disclosure is to provide a shutdown method of a portable nebulizer, including: providing a medicine storage device configured to contain predetermined medical fluid; providing a sensing device located in the medicine storage device; providing a nebulization device and a driving device, the nebulization device nebulizing the predetermined medical liquid in the medicine storage device according to a driving signal provided by the driving device and simultaneously transmitting a sensing signal to the sensing device; and providing a processing device electrically connected to the sensing device and the driving device, and collecting electrical information of the sensing signal and electrical information of the driving signal, so as to calculate and generate medical fluid volume indication information; the processing device is configured to determine whether or not to execute a shutdown procedure according to a relationship between the medical fluid volume indication information and a predetermined value range.

Therefore, according to the portable nebulizer and the shutdown method of the portable nebulizer provided by the present disclosure, even if the predetermined medical fluid in the medicine storage device has been used up and foam remains in the medicine storage device, the processing device will not misjudge that the predetermined medical fluid is still present in the medicine storage device and allow the nebulization device to continue to vibrate. In this way, the nebulization device will not continue to vibrate when the predetermined medical fluid is used up, thereby preventing the nebulization device from being reduced in service life or even damaged.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a functional block diagram of a portable nebulizer according to one embodiment of the present disclosure;

FIG. 2 is a flowchart of a shutdown method of the portable nebulizer according to one embodiment of the present disclosure;

FIG. 3 is a diagram illustrating a relationship between a sensing signal and a remaining amount of predetermined medical fluid according to one embodiment of the present disclosure;

FIG. 4 is a diagram illustrating a relationship between a driving signal and the remaining amount of the predetermined medical fluid according to one embodiment of the present disclosure; and

FIG. 5 is a diagram illustrating a relationship between medical fluid volume indication information and the remaining amount of the predetermined medical fluid according to one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

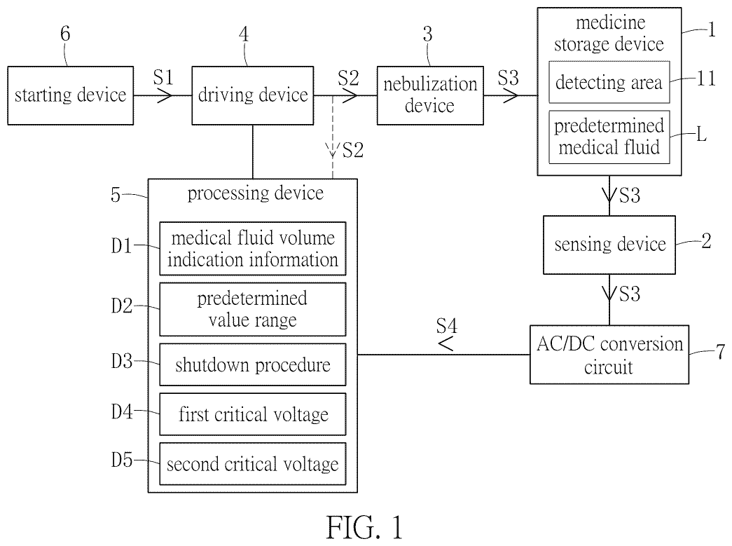

FIG. 1 is a functional block diagram of a portable nebulizer according to one embodiment of the present disclosure. Referring to FIG. 1, the portable nebulizer includes a medicine storage device 1, a sensing device 2, a nebulization device 3, a driving device 4, a processing device 5, a starting device 6, and an AC/DC (alternating current/direct current) conversion circuit 7. The medicine storage device 1 is configured to contain predetermined medical fluid L, the sensing device 2 and the nebulization device 3 are electrically connected to the medicine storage device 1, and the sensing device 2 is located in the medicine storage device 1. The sensing device 2 is further electrically connected to one side of the AC/DC conversion circuit 7, and another side of the AC/DC conversion circuit 7 is electrically connected to the processing device 5. The driving device 4 is electrically connected to the nebulization device 3 and the processing device 5, and the starting device 6 is electrically connected to the driving device 4. It should be noted that the aforementioned starting device 6 may preferably be a power supply device, but the present disclosure is not limited thereto.

The processing device 5 is, for instance, any one of an embedded controller, an application specific integrated circuit (ASIC), a field programmable gate array (FPGA), a micro control unit (MCU), a microprocessor, and a system on chip (SOC), or any combination thereof, or may be implemented with other components, firmware, and software, but the present disclosure is not limited thereto.

The starting device 6 transmits a starting signal S1 to the driving device 4. The driving device 4 is a boost conversion circuit and/or a boost component. The driving device 4 amplifies the starting signal S1 to generate a driving signal S2 and simultaneously transmits the driving signal S2 to the nebulization device 3. For instance, the starting signal S1 is an AC voltage signal, and a driving voltage of the driving signal S2 is N times a starting voltage of the starting signal S1, where N is between 1.5 and 5.

In one embodiment, the nebulization device 3 is a nebulization module. The frequency of the driving signal S2 transmitted by the driving device 4 to the nebulization device 3 must be within an allowed frequency range, and the voltage of the driving signal S2 must be within an allowed voltage range. Therefore, the driving signal S2 can drive the nebulization device 3 (i.e., the nebulization module) to vibrate. When the nebulization device 3 vibrates, the predetermined medical fluid L in the medicine storage device 1 will be converted into aerosol fluid.

Next, the converted aerosol fluid passes through a nebulizing chamber (not shown) of the portable nebulizer and is mixed with the air flowing in from the outside. After the aerosol fluid is mixed with the air flowing in from the outside, the mixture can be sprayed out through a nozzle of the nebulizing chamber for a user to absorb, thereby achieving a therapeutic effect. A remaining amount of the predetermined medical fluid L changes with the vibration time of the nebulization device 3, and the driving voltage of the driving signal S2 changes with the remaining amount of the predetermined medical fluid L.

The medicine storage device 1 includes a detecting area 11, and the sensing device 2 and the nebulization device 3 are located in the detecting area 11. The nebulization device 3 is configured to nebulize the predetermined medical fluid L in the medicine storage device 1 and simultaneously transmit a sensing signal S3 to the sensing device 2.

In one embodiment, the sensing device 2 can be a metal sensing sheet, and the metal sensing sheet is located in the detecting area 11 (not shown). The metal sensing sheet detects a radio frequency signal (i.e., the sensing signal S3) emitted by the nebulization device 3 in the medicine storage device 1 through electrical signal conduction, and the predetermined medical fluid L or air in the medicine storage device 1 is used as the transmission medium. Since the attenuation of the radio frequency signal when the radio frequency signal is transmitted in liquid will be much lower than that when the radio frequency signal is transmitted in air (in other words, the attenuation of the radio frequency signal when the radio frequency signal is transmitted through liquid is lower, and the attenuation of radio frequency signal when the radio frequency signal is transmitted through air is higher), when the predetermined medical fluid L is present in the medicine storage device 1, the radio frequency signal emitted by the nebulization device 3 is transmitted in the liquid, and the radio frequency signal detected by the metal sensing sheet will be substantially complete. On the contrary, when the predetermined medical fluid L in the medicine storage device 1 is used up, the radio frequency signal emitted by the nebulization device 3 will only be transmitted in the air, and the radio frequency signal detected by the metal sensor will be weak due to rapid attenuation in the air. Therefore, the intensity of the radio frequency signal emitted by the nebulization device 3 after the radio frequency signal is received by the sensing device 2 can be used to determine whether or not the predetermined medical fluid L in the medicine storage device 1 has been used up.

When the sensing device 2 receives the sensing signal S3 from the nebulization device 3 through the predetermined medical fluid L or air, the sensing device 2 first transmits the sensing signal S3 to the AC/DC conversion circuit 7 for conversion processing, and then transmits a sensing signal S4 that is converted to the processing device 5, such that the processing device 5 obtains the sensing signal S4.

In the present disclosure, the processing device 5 includes a memory (not shown), and the memory can be any one of a programmable read-only memory (PROM), an erasable programmable read-only memory (EPROM), a flash memory, or any combination thereof, but the disclosure is not limited thereto.

Each time the processing device 5 obtains the sensing signal S4 and the driving signal S2, the processing device 5 will collect electrical information of the sensing signal S4 and electrical information of the driving signal S2 to calculate and generate medical fluid volume indication information D1, and store the medical fluid volume indication information D1 in the memory.

The memory of the processing device 5 pre-stores a predetermined value range D2 and a shutdown procedure D3. The processing device 5 is configured to determine whether or not to execute the shutdown procedure D3 based on a relationship between the medical fluid volume indication information D1 and the predetermined value range D2.

In one embodiment, the electrical information of the sensing signal S4 is the sensing voltage of the sensing signal S4, and the electrical information of the driving signal S2 is the driving voltage of the driving signal S2. When the processing device 5 collects the sensing voltage of the sensing signal S4 and the driving voltage of the driving signal S2, the processing device 5 divides the sensing voltage of the sensing signal S4 by the driving voltage of the driving signal S2 to calculate a voltage ratio, and the voltage ratio is the medical fluid volume indication information D1. Next, the processing device 5 determines whether or not the medical fluid volume indication information D1 is within the predetermined value range D2. When the processing device 5 determines that the medical fluid volume indication information D1 is within the predetermined value range D2, the processing device 5 executes the shutdown procedure D3 to stop the vibration of the nebulization device 3. On the contrary, when the processing device 5 determines that the medical fluid volume indication information D1 is not within the predetermined value range D2, the nebulization device 3 continues to vibrate.

The memory of the processing device 5 further stores a first critical voltage D4. When the processing device 5 collects the sensing voltage of the sensing signal S4, the processing device 5 determines whether or not the sensing voltage of the sensing signal S4 is less than or equal to the first critical voltage D4. When the processing device 5 determines that the sensing voltage of the sensing signal S4 is less than or equal to the first critical voltage D4, the processing device 5 executes the shutdown procedure D3 to stop the nebulization device 3 from vibrating.

The memory of the processing device 5 further pre-stores a second critical voltage D5. When the processing device 5 collects the driving voltage of the driving signal S2, the processing device 5 determines whether or not the driving voltage of the driving signal S2 is greater than the second critical voltage D5. When the processing device 5 determines that the driving voltage of the driving signal S2 is greater than the second critical voltage D5, the processing device 5 executes the shutdown procedure D3 to stop the vibration of the nebulization device 3.

It can be learned from the above-mentioned description that, in the present disclosure, when: (1) the voltage ratio between the sensing signal S4 and the driving signal S2 is within the predetermined value range D2, or (2) the sensing voltage of the sensing signal S4 is less than or equal to the first critical voltage D4, or (3) the driving voltage of the driving signal S2 is greater than the second critical voltage D5, the shutdown procedure D3 can be triggered to stop the vibration of the nebulization device 3.

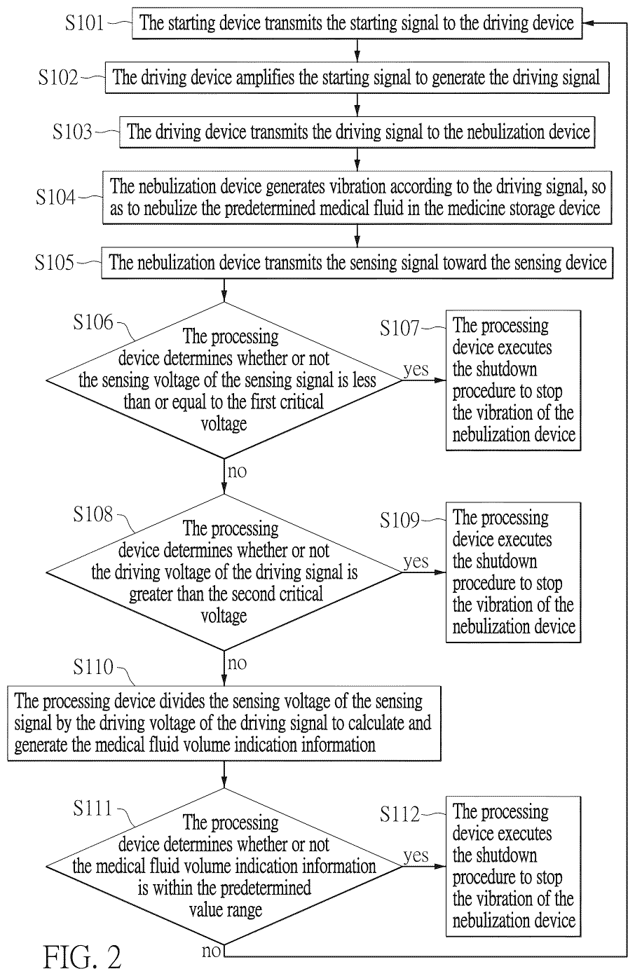

FIG. 2 is a flowchart of a shutdown method of a portable nebulizer according to one embodiment of the present disclosure. Referring to FIG. 2, in step S101, the starting device 6 transmits the starting signal S1 to the driving device 4.

In step S102, the driving device 4 amplifies the starting signal S1 to generate the driving signal S2. Specifically, the driving device 4 can be, for instance, a boost conversion circuit.

In step S103, the driving device 4 transmits the driving signal S2 to the nebulization device 3.

In step S104, the nebulization device 3 generates vibration according to the driving signal S2, so as to nebulize the predetermined medical fluid L in the medicine storage device 1.

In one embodiment, the nebulization device 3 is a nebulization module. The frequency of the driving signal S2 must be within the allowed frequency range, and the driving voltage of the driving signal S2 must be within the allowed voltage range, such that the driving signal S2 can drive the nebulizer device 3 to vibrate.

In step S105, the nebulization device 3 transmits the sensing signal S3 toward the sensing device 2. Specifically, the sensing voltage of the sensing signal S3 changes with the remaining amount of the predetermined medical fluid L. When the remaining amount of the predetermined medical fluid L decreases, the sensing voltage of the sensing signal S3 is lowered accordingly.

In step S106, the processing device 5 determines whether or not the sensing voltage of the sensing signal S4 is less than or equal to the first critical voltage D4.

When the processing device 5 determines that the sensing voltage of the sensing signal S4 is less than or equal to the first critical voltage D4, step S107 is performed.

When the processing device 5 determines that the sensing voltage of the sensing signal S4 is not less than or equal to the first critical voltage D4, step S108 is performed.

In step S107, the processing device 5 executes the shutdown procedure D3 to stop the vibration of the nebulization device 3.

In step S108, the processing device 5 determines whether or not the driving voltage of the driving signal S2 is greater than the second critical voltage D5.

When the processing device 5 determines that the driving voltage of the driving signal S2 is greater than the second critical voltage D5, step S109 is performed.

When the processing device 5 determines that the driving voltage of the driving signal S2 is not greater than the second critical voltage D5, step S110 is performed.

In step S109, the processing device 5 executes the shutdown procedure D3 to stop the vibration of the nebulization device 3.

In step S110, the processing device 5 divides the sensing voltage of the sensing signal S4 by the driving voltage of the driving signal S2 to calculate and generate the medical fluid volume indication information D1.

In step S111, the processing device 5 determines whether or not the medical fluid volume indication information D1 is within the predetermined value range D2.

When the processing device 5 determines that the medical fluid volume indication information D1 is within the predetermined value range D2, step S112 is performed.

When the processing device 5 determines that the medical fluid volume indication information D1 is not within the predetermined value range D2, the shutdown method returns to step S101.

In step S112, the processing device 5 executes the shutdown procedure D3 to stop the vibration of the nebulization device 3.

| Vibration | Remaining | | | Medical |
|---|---|---|---|---|
| time of | amount of | | | fluid volume |
| nebulization | predetermined | Sensing | Driving | indication |
| device 3 | medical fluid L | signal S4 | signal S2 | information |
| (seconds, s) | (grams, g) | (volt, V) | (volt, V) | D1 |
| 0 | 2 | 2.7391 | 1.7603 | 1.5561 |
| 21.08 | 0.1602 | 2.4343 | 1.9578 | 1.2434 |
| 22.03 | 0.1306 | 2.6662 | 2.2282 | 1.1965 |

For instance, the initial amount of the predetermined medical fluid L is 2 g, and an upper limit and a lower limit of the predetermined value range D2 are 1.3 and 0, respectively. The first critical voltage D4 is 2.4 V, and the second critical voltage D5 is 2.2 V.

When the nebulization device 3 continues to vibrate and the vibration time reaches 21.08 s, the sensing voltage (2.4343 V) of the sensing signal S4 is not less than or equal to the first critical voltage D4 (2.4 V), and the driving voltage (1.7603 V) of the driving signal S2 is not greater than the second critical voltage D5 (2.2 V). However, at this time, the voltage ratio of the sensing voltage of the sensing signal S4 to the driving voltage of the driving signal S2 is already within the predetermined value range D2. At this time, the condition for the medical fluid volume indication information D1 to trigger shutdown has been met.

When the vibration time of the nebulization device 3 reaches 22.03 s, the sensing voltage (2.6662 V) of the sensing signal S4 is not less than or equal to the first critical voltage D4 (2.4 V), but the driving voltage (2.2282 V) of the driving signal S2 is already greater than the second critical voltage D5 (2.2 V). At this time, the condition for the driving signal S2 to trigger the shutdown has been met, but the condition for the sensing signal S4 to trigger the shutdown has not yet been met.

It can be learned that through the above restrictions, the time point at which the medical fluid volume indication information D1 triggers shutdown is earlier than the time point at which the driving signal S2 triggers shutdown, and the time point at which the driving signal S2 triggers shutdown is earlier than the time point at which the sensing signal S4 triggers the shutdown. This effectively avoids the situation where the nebulization device 3 continues to vibrate when the predetermined medical fluid L is used up.

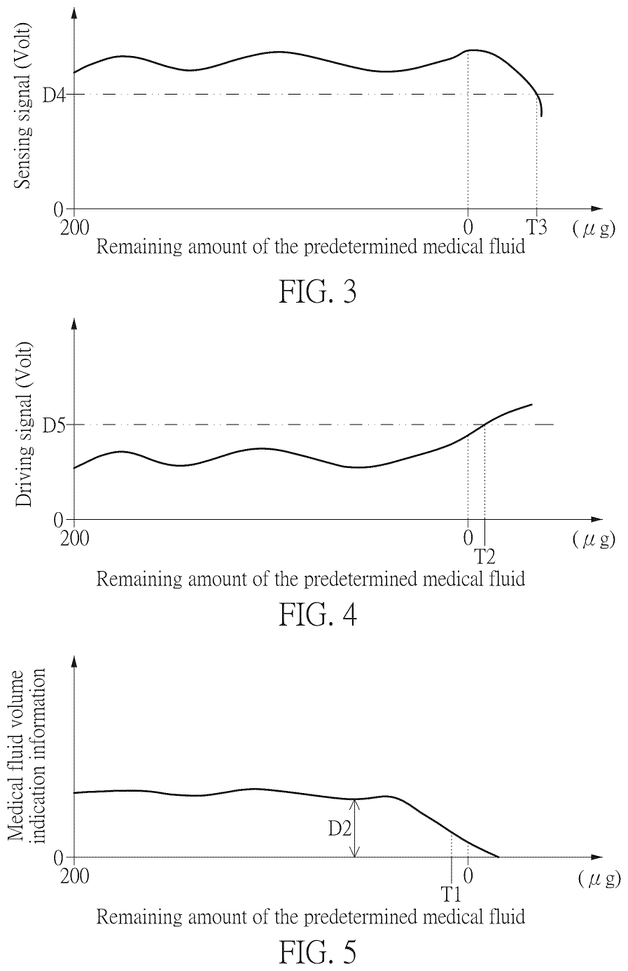

FIG. 3 is a diagram illustrating a relationship between a sensing signal S4 and a remaining amount of the predetermined medical fluid L in the medicine storage device 1 according to one embodiment of the present disclosure. Referring to FIG. 3, the initial amount of the predetermined medical fluid L in the medicine storage device 1 is 200 μg, and the remaining amount of the predetermined medical fluid L decreases as the vibration time of the nebulization device 3 increases. When the predetermined medical fluid L is used up, the sensing voltage of the sensing signal S4 is still not less than or equal to the first critical voltage D4 at this time; therefore, the condition for the sensing signal S4 to trigger shutdown has not yet been met.

For instance, when the predetermined medical fluid L has been used up and a plurality of foam appear in the medicine storage device 1, the processing device 5 may misjudge that the predetermined medical fluid L in the medicine storage device 1 has not been used up due to the foam, resulting in the nebulization device 3 continues to vibrate when the predetermined medical fluid L is used up, which further leads to a reduction in the service life of the nebulization device 3 or even damage dealt to the nebulization device 3. It can be learned that if the sensing signal S4 is used as the only condition for triggering shutdown, the nebulization device 3 may continue to vibrate even when the predetermined medical fluid L is used up.

FIG. 4 is a diagram illustrating a relationship between a driving signal S2 and the remaining amount of the predetermined medical fluid L in the medicine storage device 1 according to one embodiment of the present disclosure. Referring to FIG. 4, although the predetermined medical fluid L is still present in the medicine storage device 1, since the predetermined medical fluid L is about to be used up, the driving voltage of the driving signal S2 will rise. However, the predetermined medical fluid L is likely to be used up during the rising of the driving voltage of the driving signal S2, but the driving voltage of the driving signal S2 is not yet greater than the second critical voltage D5. Therefore, the nebulization device 3 may still vibrate when the predetermined medical fluid L is used up. It can be learned that, if the driving signal S2 is only used as the condition for triggering shutdown, the nebulization device 3 may still continue to vibrate when the predetermined medical fluid L is used up.

FIG. 5 is a diagram illustrating a relationship between the medical fluid volume indication information D1 and the remaining amount of the predetermined medical fluid L in the medicine storage device 1 according to one embodiment of the present disclosure. Referring to FIG. 5, when the predetermined medical fluid L in the medicine storage device 1 is about to be used up, the voltage ratio between the sensing voltage of the sensing signal S4 and the driving voltage of the driving signal S2 will be within the predetermined value range D2 and meet the conditions for the medical fluid volume indication information D1 to trigger shutdown. It can be learned that, using the medical fluid volume indication information D1 as a condition for triggering shutdown can effectively avoid the situation where the nebulization device 3 continues to vibrate when the predetermined medical fluid L is used up.

Referring to FIG. 3 to FIG. 5, a first time T1 when the medical fluid volume indication information D1 triggers the shutdown is earlier than a second time T2 when the drive signal S2 triggers the shutdown, and the second time T2 when the drive signal S2 triggers the shutdown is earlier than a third time T3 when the radio frequency signal (i.e., the sensing signal S4) triggers the shutdown. It can be learned that, compared with relying on the sensing voltage of the sensing signal S4 as a condition for triggering shutdown or relying on the driving voltage of the driving signal S2 as a condition for triggering shutdown, relying on the medical fluid volume indication information D1 as the condition for triggering shutdown can more effectively prevent the nebulization device 3 from continuing to vibrate even when the predetermined medical fluid L is used up.

### Beneficial Effects of the Embodiments

In conclusion, according to the portable nebulizer and the shutdown method of the portable nebulizer provided by the present disclosure, even if the predetermined medical fluid in the medicine storage device has been used up and a plurality of foam appears in the medicine storage device, the processing device will not misjudge that the predetermined medical fluid is still present in the medicine storage device and allow the nebulization device to continue to vibrate. In this way, the nebulization device will not continue to vibrate when the predetermined medical fluid is used up, thereby preventing the nebulization device from being reduced in service life or even damaged.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A portable nebulizer, comprising:
a medicine storage device configured to contain predetermined medical fluid;
a sensing device located in the medicine storage device;
a nebulization device configured to nebulize the predetermined medical fluid and simultaneously transmit a sensing signal to the sensing device;
a driving device electrically connected to the nebulization device and transmitting a driving signal to the nebulization device; and
a processing device electrically connected to the sensing device and the driving device, and collecting electrical information of the sensing signal and electrical information of the driving signal, so as to calculate and generate medical fluid volume indication information;
wherein the processing device is configured to determine whether or not to execute a shutdown procedure according to a relationship between the medical fluid volume indication information and a predetermined value range.

2. The portable nebulizer according to claim 1, wherein, when the processing device collects the electrical information of the sensing signal and the electrical information of the driving signal to calculate the medical fluid volume indication information, the processing device is further configured to:
collect a sensing voltage of the sensing signal;
collect a driving voltage of the driving signal; and
divide the sensing voltage of the sensing signal by the driving voltage of the driving signal, so as to calculate and generate the medical fluid volume indication information.

3. The portable nebulizer according to claim 2, wherein the driving voltage of the driving signal changes with a remaining amount of the predetermined medical fluid.

4. The portable nebulizer according to claim 1, further comprising a starting device, wherein the starting device is electrically connected to the driving device, and the driving device is a boost conversion circuit and/or a boost component.

5. The portable nebulizer according to claim 2, wherein, when a frequency of the driving signal is within an allowed frequency range and the driving voltage of the driving signal is within an allowed voltage range, the nebulization device generates vibration.

6. A shutdown method of a portable nebulizer, comprising:
providing a medicine storage device configured to contain predetermined medical fluid;
providing a sensing device located in the medicine storage device;
providing a nebulization device and a driving device, wherein the nebulization device nebulizes the predetermined medical liquid in the medicine storage device according to a driving signal provided by the driving device and simultaneously transmits a sensing signal to the sensing device; and
providing a processing device electrically connected to the sensing device and the driving device, and collecting electrical information of the sensing signal and electrical information of the driving signal, so as to calculate and generate medical fluid volume indication information;
wherein the processing device is configured to determine whether or not to execute a shutdown procedure according to a relationship between the medical fluid volume indication information and a predetermined value range.

7. The shutdown method according to claim 6, wherein the process of collecting the electrical information of the sensing signal and the electrical information of the driving signal to calculate and generate the medical fluid volume indication information includes:
collecting a sensing voltage of the sensing signal;
collecting a driving voltage of the driving signal; and
dividing the sensing voltage of the sensing signal by the driving voltage of the driving signal, so as to calculate and generate the medical fluid volume indication information.

8. The shutdown method according to claim 7, wherein the driving voltage of the driving signal changes with a remaining amount of the predetermined medical fluid; when a frequency of the driving signal is within an allowed frequency range and the driving voltage of the driving signal is within an allowed voltage range, the nebulization device generates vibration.

9. The shutdown method according to claim 7, wherein, when the processing device determines that the sensing voltage of the sensing signal is less than or equal to a first critical voltage, the processing device executes the shutdown procedure; when the processing device determines that the driving voltage of the driving signal is greater than a second critical voltage, the processing device executes the shutdown procedure.

10. The shutdown method according to claim 9, wherein the processing device determines at a first time that the medical fluid volume indication information is within the predetermined value range, the processing device determines at a second time that the driving voltage of the driving signal is greater than the second critical voltage, the processing device determines at a third time that the sensing voltage of the sensing signal is less than or equal to the first critical voltage, and the first time is earlier than the second time, and the second time is earlier than the third time.
